SEQUENCE LISTING (79 sequences)

SEQ ID NO: 1 - DNA, 96 bp, Artificial Sequence
  Description of Artificial Sequence Synthetic oligonucleotide
tttggtccttgtcttatgtccagaatgcnnnnnnnnnnnnnnnnnnnnnnnnnnnnnnnnnnnnnnnnatttctcctactgggataggtggattat

SEQ ID NO: 2 - DNA, 28 bp, Artificial Sequence
  Description of Artificial Sequence Synthetic primer
ataatccacctatcccagtaggagaaat

SEQ ID NO: 3 - DNA, 28 bp, Artificial Sequence
  Description of Artificial Sequence Synthetic oligonucleotide
tttggtccttgtcttatgtccagaatgc

SEQ ID NO: 4 - DNA, 28 bp, Artificial Sequence
  Description of Artificial Sequence Synthetic oligonucleotide
atttctcctactgggataggtggattat

SEQ ID NO: 5 - DNA, 66 bp, Artificial Sequence
  Description of Artificial Sequence Synthetic oligonucleotide
aaaccaggaacagaatacaggtcttacggcggatcgtgatttctcctactgggataggtggattat

SEQ ID NO: 6 - DNA, 66 bp, Artificial Sequence
  Description of Artificial Sequence Synthetic oligonucleotide
aaaccaggaacagaatacaggtcttacgggctacaggcatttctcctactgggataggtggattat

SEQ ID NO: 7 - DNA, 64 bp, Artificial Sequence
  Description of Artificial Sequence Synthetic oligonucleotide
aaaccaggaacagaatacaggtcttacggtggggcaatttctcctactgggataggtggattat

SEQ ID NO: 8 - DNA, 10 bp, Artificial Sequence
  Description of Artificial Sequence Synthetic oligonucleotide
gcggatcgtg

SEQ ID NO: 9 - DNA, 10 bp, Artificial Sequence
  Description of Artificial Sequence Synthetic oligonucleotide
ggctacaggc

SEQ ID NO: 10 - DNA, 17 bp, Artificial Sequence
  Description of Artificial Sequence Synthetic oligonucleotide
cttacggtggggcaatt

SEQ ID NO: 11 - DNA, 17 bp, Artificial Sequence
  Description of Artificial Sequence Synthetic oligonucleotide
atttgccccaccgtaag

SEQ ID NO: 12 - DNA, 28 bp, Artificial Sequence
  Description of Artificial Sequence Synthetic oligonucleotide
tttggtccttgtcttatgtccagaatgc

SEQ ID NO: 13 - DNA, 28 bp, Artificial Sequence
  Description of Artificial Sequence Synthetic oligonucleotide
atttctcctactgggataggtggattat

SEQ ID NO: 14 - DNA, 173 bp, Artificial Sequence
  Description of Artificial Sequence Synthetic oligonucleotide
ggcttcataacttgagactgcggtacatagggaagcaggctacccgggcttgatctggggcacaaaccccttccactccactcgagccccgggctaccctgccagctgagtcgcctgacacccgcaagcgcccacacaggattccacacgtgatcctaagacctctaaagccc

SEQ ID NO: 15 - DNA, 31 bp, Artificial Sequence
  Description of Artificial Sequence Synthetic oligonucleotide
acccgggcttgatctggggcacaaacccctt

SEQ ID NO: 16 - DNA, 184 bp, Artificial Sequence
  Description of Artificial Sequence Synthetic oligonucleotide
gaattcggcttcataacttgagactgcggtacatagggaagcaggctacccgggcttgatctggggcacaaaccccttccactccactcgagccccgggctaccctgccagctgagtcgcctgacacccgcaagggcccacacaggattccacacgtgatcctaagacctctaaagccgaattc

SEQ ID NO: 17 - DNA, 145 bp, Artificial Sequence
  Description of Artificial Sequence Synthetic oligonucleotide
gcccttagtttagtctcagagaacccatggtaacaaaggtaggcataatataacatgcctttaagctgagtgttctggccagctttatgccaatttgacagtcatttaggaagaggggacctcctttgagaaaatatgcaagggc

SEQ ID NO: 18 - DNA, 40 bp, Artificial Sequence
  Description of Artificial Sequence Synthetic oligonucleotide
gcgaatcccaccaaataagtcaattcacaagtacgcgcca

SEQ ID NO: 19 - DNA, 40 bp, Artificial Sequence
  Description of Artificial Sequence Synthetic oligonucleotide
ctacagcagaatctgtgtcgtctgtacggggatggcgtac

SEQ ID NO: 20 - DNA, 40 bp, Artificial Sequence
  Description of Artificial Sequence Synthetic oligonucleotide
taggtaggacaaggtgctgcaaggacgtagagtggaggcg

SEQ ID NO: 21 - DNA, 20 bp, Artificial Sequence
  Description of Artificial Sequence Synthetic oligonucleotide
cgaattacccatcagctcat

SEQ ID NO: 22 - DNA, 40 bp, Artificial Sequence
  Description of Artificial Sequence Synthetic oligonucleotide
atggaggctgcgctcaccatgtagcccccacgctagtcgt

SEQ ID NO: 23 - DNA, 40 bp, Artificial Sequence
  Description of Artificial Sequence Synthetic oligonucleotide
accgggaatgtcctcacgttcccttaaacatcatgcaccg

SEQ ID NO: 24 - DNA, 40 bp, Artificial Sequence
  Description of Artificial Sequence Synthetic oligonucleotide
cataatcggacaagatttaactgacatagtgcaagattgc

SEQ ID NO: 25 - DNA, 40 bp, Artificial Sequence
  Description of Artificial Sequence Synthetic oligonucleotide
acaccctgcgcttactgaatactagtgccatcaccttcgg

SEQ ID NO: 26 - DNA, 40 bp, Artificial Sequence
  Description of Artificial Sequence Synthetic oligonucleotide
ttagagaacctcggtgcatcctgccaactactgggcctcg

SEQ ID NO: 27 - DNA, 40 bp, Artificial Sequence
  Description of Artificial Sequence Synthetic oligonucleotide
aatgaacattcggatagcactcgataactattaacggcgc

SEQ ID NO: 28 - DNA, 40 bp, Artificial Sequence
  Description of Artificial Sequence Synthetic oligonucleotide
gacaatctcacaagaatgtagctggattctccgcccatac

SEQ ID NO: 29 - DNA, 40 bp, Artificial Sequence
  Description of Artificial Sequence Synthetic oligonucleotide
tcacagttgctgtggagtgagtaaccgtcactcaaccggg

SEQ ID NO: 30 - DNA, 40 bp, Artificial Sequence
  Description of Artificial Sequence Synthetic oligonucleotide
caggctaaaagcatcgacattaatcaatagagctaacgat

SEQ ID NO: 31 - DNA, 40 bp, Artificial Sequence
  Description of Artificial Sequence Synthetic oligonucleotide
cgccatcgacagcttttactgtgagagacggtacaagcgc

SEQ ID NO: 32 - DNA, 40 bp, Artificial Sequence
  Description of Artificial Sequence Synthetic oligonucleotide
gaaagctagtatacctgacatagatctcactaccccactg

SEQ ID NO: 33 - DNA, 40 bp, Artificial Sequence
  Description of Artificial Sequence Synthetic oligonucleotide
cgccgtgggatggattcataaaccgatgtagtttaagtgc

SEQ ID NO: 34 - DNA, 40 bp, Artificial Sequence
  Description of Artificial Sequence Synthetic oligonucleotide
tgccgggaaatatcacataacacccttatgcctcctaccc

SEQ ID NO: 35 - DNA, 40 bp, Artificial Sequence
  Description of Artificial Sequence Synthetic oligonucleotide
ctgtgatgaaacgtaattgtccactgagaggtgacttcgc

SEQ ID NO: 36 - DNA, 40 bp, Artificial Sequence
  Description of Artificial Sequence Synthetic oligonucleotide
taggcagtaacagtagcgaactgattgtgcaaatgtaggg

SEQ ID NO: 37 - DNA, 40 bp, Artificial Sequence
  Description of Artificial Sequence Synthetic oligonucleotide
aaactctgacatatagttagtcgtgaagctgcttgacgcc

SEQ ID NO: 38 - DNA, 40 bp, Artificial Sequence
  Description of Artificial Sequence Synthetic oligonucleotide
tccctgccacccaatacgtcgggtaagcattgaacgcggc

SEQ ID NO: 39 - DNA, 80 bp, Artificial Sequence
  Description of Artificial Sequence Synthetic oligonucleotide
cgcagagcatcgtaatattggcgatagcactaagtccgcctgtccatcgacattctagtcccatccaatgtaaagcacag

SEQ ID NO: 40 - DNA, 40 bp, Artificial Sequence
  Description of Artificial Sequence Synthetic oligonucleotide
caatgggcttagcactgttacactggacgaatgcgcggta

SEQ ID NO: 41 - DNA, 40 bp, Artificial Sequence
  Description of Artificial Sequence Synthetic oligonucleotide
ctccccatgttaccaattgtcgaaccgagctacccagatg

SEQ ID NO: 42 - DNA, 40 bp, Artificial Sequence
  Description of Artificial Sequence Synthetic oligonucleotide
tagaccgctcgtacaggagctgatgcaaagtgagctatgt

SEQ ID NO: 43 - DNA, 40 bp, Artificial Sequence
  Description of Artificial Sequence Synthetic oligonucleotide
cgagactttctgtgaacccacatcatcgaggtgtgaccgc

SEQ ID NO: 44 - DNA, 40 bp, Artificial Sequence; identical to SEQ ID NO: 35
  Description of Artificial Sequence Synthetic oligonucleotide

SEQ ID NO: 45 - DNA, 40 bp, Artificial Sequence
  Description of Artificial Sequence Synthetic oligonucleotide
cgaatacacaacaatggcagcaactctgtatgaatggggg

SEQ ID NO: 46 - DNA, 40 bp, Artificial Sequence; identical to SEQ ID NO: 32
  Description of Artificial Sequence Synthetic oligonucleotide

SEQ ID NO: 47 - DNA, 40 bp, Artificial Sequence
  Description of Artificial Sequence Synthetic oligonucleotide
cctctccctcagcacacggctcaactcagtgtagccagca

SEQ ID NO: 48 - DNA, 40 bp, Artificial Sequence
  Description of Artificial Sequence Synthetic oligonucleotide
cgccacatatggattacgtacgcgcaatacaagggaatgc

SEQ ID NO: 49 - DNA, 40 bp, Artificial Sequence
  Description of Artificial Sequence Synthetic oligonucleotide
tatcctagaagaccaccgcgcccatctatggattgagttg

SEQ ID NO: 50 - DNA, 40 bp, Artificial Sequence
  Description of Artificial Sequence Synthetic oligonucleotide
agatatagcagatcaaaagattaaccatgcgcttaccgcc

SEQ ID NO: 51 - DNA, 40 bp, Artificial Sequence
  Description of Artificial Sequence Synthetic oligonucleotide
ccaatctgaatcccgcatgtattcatccataacgctcgcg

SEQ ID NO: 52 - DNA, 40 bp, Artificial Sequence
  Description of Artificial Sequence Synthetic oligonucleotide
tctaatggccttacctttactatcttaaggctatcggggc

SEQ ID NO: 53 - DNA, 40 bp, Artificial Sequence
  Description of Artificial Sequence Synthetic oligonucleotide
acctgatcactgttagtccacacagtttgtggggaccatc

SEQ ID NO: 54 - DNA, 41 bp, Artificial Sequence
  Description of Artificial Sequence Synthetic oligonucleotide
agttacagctcacgtccagaaacccctggtcttctcccgct

SEQ ID NO: 55 - DNA, 40 bp, Artificial Sequence
  Description of Artificial Sequence Synthetic oligonucleotide
cggaatcctagtcgtttgacataagaaacgcagtatttgg

SEQ ID NO: 56 - DNA, 40 bp, Artificial Sequence
  Description of Artificial Sequence Synthetic oligonucleotide
attagctaagagtgatgtcgtatattctataggtgcggtc

SEQ ID NO: 57 - DNA, 38 bp, Artificial Sequence
  Description of Artificial Sequence Synthetic oligonucleotide
aaagcggagaacacggttgaggtgggacagagggacac

SEQ ID NO: 58 - DNA, 40 bp, Artificial Sequence
  Description of Artificial Sequence Synthetic oligonucleotide
cgcgatttgaagatttatgtcctaagtcatggtctgtgtt

SEQ ID NO: 59 - DNA, 40 bp, Artificial Sequence
  Description of Artificial Sequence Synthetic oligonucleotide
tcgttgatctcgtaacagaccaaggagacggttcataccg

SEQ ID NO: 60 - DNA, 40 bp, Artificial Sequence
  Description of Artificial Sequence Synthetic oligonucleotide
ttacaagaagggccagaagaggaatatcgcaatcatgttt

SEQ ID NO: 61 - DNA, 40 bp, Artificial Sequence
  Description of Artificial Sequence Synthetic oligonucleotide
tctggtgacttgggctgtatgtagcctcgggttgctgcac

SEQ ID NO: 62 - DNA, 40 bp, Artificial Sequence
  Description of Artificial Sequence Synthetic oligonucleotide
gcccgtacgtcacccgaacagcgggctatctatacactgc

SEQ ID NO: 63 - DNA, 40 bp, Artificial Sequence
  Description of Artificial Sequence Synthetic oligonucleotide
tgctcgaatcgtcgacaaatagtattccgggccgtgagtc

SEQ ID NO: 64 - DNA, 40 bp, Artificial Sequence
  Description of Artificial Sequence Synthetic oligonucleotide
aatgtcaaataggagggcgcatttgaccccagccaagtac

SEQ ID NO: 65 - DNA, 40 bp, Artificial Sequence
  Description of Artificial Sequence Synthetic oligonucleotide
tccctaattcgttgaacaatcaagatgattttgcatttgg

SEQ ID NO: 66 - DNA, 40 bp, Artificial Sequence
  Description of Artificial Sequence Synthetic oligonucleotide
agcgttgtaatgtttcatatagtgtcctagggaacttgtc

SEQ ID NO: 67 - DNA, 40 bp, Artificial Sequence
  Description of Artificial Sequence Synthetic oligonucleotide
cgccgtacccggttacccagcccagctgtagctcggagca

SEQ ID NO: 68 - DNA, 40 bp, Artificial Sequence
  Description of Artificial Sequence Synthetic oligonucleotide
cagctgaggtatgttagcgtgtcggtatagtgtgggcggt

SEQ ID NO: 69 - DNA, 40 bp, Artificial Sequence; identical to SEQ ID NO: 67
  Description of Artificial Sequence Synthetic oligonucleotide

SEQ ID NO: 70 - DNA, 40 bp, Artificial Sequence
  Description of Artificial Sequence Synthetic oligonucleotide
aacgaatacacggctaggtcgtgattagtgaagtgtagtt

SEQ ID NO: 71 - DNA, 40 bp, Artificial Sequence
  Description of Artificial Sequence Synthetic oligonucleotide
aaacgataagcgttcatccgctataatcgagtcggatctc

SEQ ID NO: 72 - DNA, 40 bp, Artificial Sequence
  Description of Artificial Sequence Synthetic oligonucleotide
ggtcaggatgcggaatagccagagatacagatgatagtgt

SEQ ID NO: 73 - DNA, 40 bp, Artificial Sequence
  Description of Artificial Sequence Synthetic oligonucleotide
caaatgagcatgtgaccggtattttcacgctcggcattag

SEQ ID NO: 74 - DNA, 40 bp, Artificial Sequence
  Description of Artificial Sequence Synthetic oligonucleotide
ccagaagacgcggtttgttatttgtggaattgccgtatgc

SEQ ID NO: 75 - DNA, 41 bp, Artificial Sequence
  Description of Artificial Sequence Synthetic oligonucleotide
tctgtggcatgtacctatcgtggaggacgattatcaggtcc

SEQ ID NO: 76 - DNA, 40 bp, Artificial Sequence
  Description of Artificial Sequence Synthetic oligonucleotide
aacgtggagggccaggccgattaggctacatcgtaggtgc

SEQ ID NO: 77 - DNA, 41 bp, Artificial Sequence
  Description of Artificial Sequence Synthetic oligonucleotide
cttcataacctgaccttcaaggtttggataagcgtttccgc

SEQ ID NO: 78 - DNA, 40 bp, Artificial Sequence
  Description of Artificial Sequence Synthetic oligonucleotide
cagaattcgtttttgctacgtgaccaggctaacgtcggcg

SEQ ID NO: 79 - DNA, 40 bp, Artificial Sequence
  Description of Artificial Sequence Synthetic oligonucleotide
tacagcgtggaagatttagtcatcgatgcccttctgcgag

(19) United States
(12) Patent Application Publication
(10) Pub. No.: US 2009/0155779 A1
(43) Pub. Date: Jun. 18, 2009

(54) APTAMERS THAT BIND TO PRION PROTEIN

(75) Inventors: Srinard Sreevatsan, St. Paul, MN (US); Kaori Nakamura, Ft. Dodge, IA (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 11/917,884

(22) PCT Filed: Jun. 15, 2006
(86) PCT No.: PCT/US2006/023673

§ 371 Date: Jul. 28, 2008

Related U.S. Application Data
(60) Provisional application No. 60/690,575, filed on Jun. 15, 2005.
(60) Provisional application No. 60/700,268, filed on Jul. 18, 2005.

(51) Int. Cl. C12Q 1/68; C07H 21/04

(52) U.S. Cl. 435/6; 536/23.1

(57) ABSTRACT

Compositions are provided in the form of nucleotide aptamerss that are capapble of binding PrP, and in some embodiments, differentially binding PrP isoforms. Also provided are methods for identifying PrP in a sample, and in some embodiments, either selectively removing PrP or PrP isoforms from a sample, or inactivating them within a sample.

### PRIORITY CLAIM

This application claims priority to U.S. Provisional Patent Application 60/690,575, filed Jun. 15, 2005, and U.S. Provisional Patent Application 60/700,268, filed Jul. 18, 2005, each of which is incorporated herein by reference, in its entirety.

### STATEMENT ON FEDERALLY FUNDED RESEARCH

The present invention was made at least in part with support from The Department of the Army, Grant NO. DAMD 17-031-0377. The United States Government has certain rights in the invention.

Transmissible spongiform encephalopathies (TSEs) are caused by unconventional transmissible agents that are called prions. TSEs essentially comprise Creutzfeldt-Jakob disease in humans (CJD), scrapie in sheep and goats, and bovine spongiform encephalopathy (BSE) in bovines. Other encephalopathies have been demonstrated in the Felidae, in mink or certain wild animals, such as deer or elk. These diseases are always fatal and, at the current time, there is no effective treatment. In TSEs, there is an accumulation of a host's protein, PrP (or prion protein), in an abnormal form, mainly in the central nervous system. The normal and disease causing forms of PrP have the same amino acid sequence, but are different in their secondary structure. Accordingly, it is desirable to have compositions that bind to PrP and variant forms thereof, including abnormally folded prion proteins, and variant forms of non-disease causing prion in humans, bovines, sheep and hamsters, and other organisms. Such compositions would desirably aid in differentiation of prion isoforms associated with specific neuropathologies or disease phenotypes, and allow differential diagnosis. Additionally, such compositions could also be relatively inexpensive and easy to use with a biological sample, such as a tissue sample.

### SUMMARY OF THE INVENTION

Disclosed herein are compositions in the form of nucleotide aptamers that are capable of binding PrP, and in some embodiments, differentially binding PrP isoforms. Also disclosed are methods for identifying PrP in a sample, and in some embodiments, either selectively removing PrP or PrP isoforms from a sample, or inactivating them within a sample.

### BRIEF DESCRIPTION OF THE DRAWINGS

FIG. 1 shows a schematic representation of SELEX methodology as disclosed according to some embodiments herein;

FIG. 2 shows results of gel shift (A) and dot blot (B) analyses for unselected aptamer (Apt) library and Apt pool after six rounds of SELEX. (A) Apt alone and Apt-PrP mixture were resolved by nondenaturing PAGE. Apt bands were visualized by ethidium bromide staining. The intensity of the Apt band decreased in the presence of PrP for the selected aptamers (arrow) indicating that the sixth-round SELEX selectively concentrated the Apt species that possessed higher affinities to PrP. (B) Signal from the unselected Apt library was too weak to capture, however, the signal was clear from the selected Apt pool against rhuPrPC23-231, indicating that the selected pool contained Apt with a higher affinity to the target PrP;

FIG. 3 shows representative structures of selected aptamers according to the invention, 1-4 (A), 1-9 (B), and 3-10 (C), derived using the program mfold (17);

FIG. 4 shows results from chemiluminescent gel shift (A) and dot blot (B) analyses for short aptamers. (A) One aptamer alone, two aptamer incubated with rhuPrPC23-231. The short aptamer sri3-10OH demonstrated multiple banding patterns, indicating a presence of secondary structures. In the presence of PrP, the bands shifted to larger molecular sizes for sri3-10OH, indicating that the aptamer bound to PrP, but that other nonspecific short aptamers did not. (B) sri3-10OH also bound to PrP by dot blot analysis, but the reverse complement sequence of sri3-10OH did not detect PrP. The positive control was biotinylated nucleotide;

FIG. 5 shows results from chemiluminescent dot blot analysis for selected aptamers that bound to PrPs. The left panel indicates positions of immobilized proteins and a control. 1: ˜ositive control for the assay (biotinylated nucleotides); 2: nonspecific protein (casein); 3: rhuPrPC90-231; 4: rhuPrPC23-231; and 5: PrP immunoprecipitated from sheep brain. The selected aptamers bound to rhuPrPc23-231, but not to rhuPrPc90-231, suggesting that the binding sites of the aptamers are located between amino acid residues 23 and 89. The aptamers reacted with recombinant and mammalian PrPC;

FIG. 6 shows a gel shift analysis showing the affinity of the selected aptamers against recombinant and mammalian PrPC. 1: aptamer alone; 2: aptamer incubated with immunoprecipitated ovine PrP; and 3: aptamer incubated with rhuPrPC23-231. In the presence of ovine PrP and rhuPrP, the aptamer bands shifted to larger molecular sizes (arrow), indicating that the aptamers bound to the PrPs;

FIG. 7 shows a dot blot analysis with selected aptamers against PrPC enriched from brain tissues of a variety of animal species. The left panel indicates the positions of the immobilized proteins and a control. The positive control was biotinylated nucleotide. Casein was used as nonspecific protein. A dot of rhuPrPC90-231 or rhuPrPC3-231 contains approximately 1 μg of protein. Sheep, cattle, pig, and deer dots contain approximately 2 μg of PrPs derived from brain tissues of apparently healthy animals;

Table 1 shows the sequences of randomized regions of selected aptamers, aptamer 3 to 10-derived short aptamers;

Table 2 shows binding concentration end points of the selected aptamers against rhu PrP^{c} 23-231, measured by concentration gradient titration;

Table 3 shows aptamer binding to PrP^{c} expressed on neuroblastoma cells, using standard cell blot assays under varying conditions

Table 4 shows counter-SELEX-developed PrP aptamerss;

FIG. 8 shows the sequence and predicted secondary structure of one PrP specific aptamer referred to as Clone 8, which includes a shorter aptamer that also shows PrP binding;

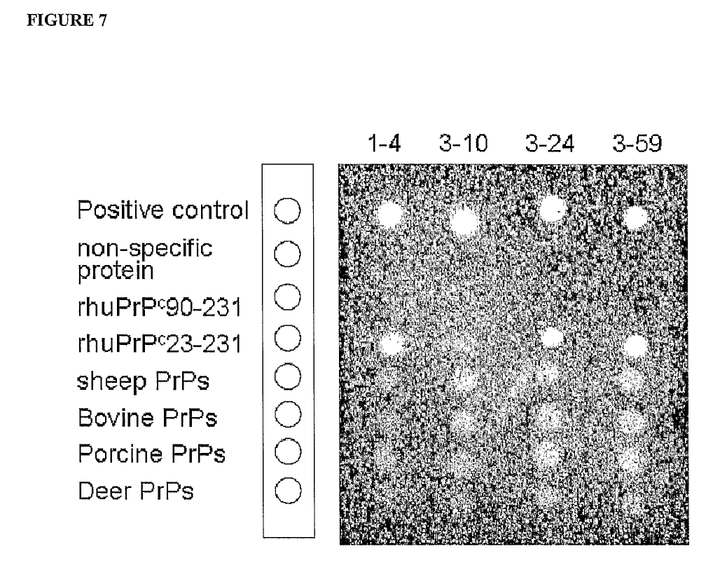

FIG. 7 shows the sequence and predicted secondary structure of another PrP specific aptamer referred to as Clone 23, which includes a shorter aptamer that also shows PrP binding

FIG. 10 shows results of Gel-shift Analysis with Aptamers Selected against PrPSc. Shown in lanes 2-4 are the reactivities of the aptamer library (SSAP40) with PrPSc before (PK−) and after proteinase K(PK+) treatment. Lanes 5-7 show aptamer amplicon after 8 rounds of SELEX leading to a clear gel shift (arrows) when reacted with PK+ PrPSc or recombinant 90-231. Lanes 8-10 show evidence of gel shift of the 8th SELEX aptamers selected against untreated PrPSc or full length recombinant 23-231. Shown in Lane 1 is a 100-bp DNA ladder.

### DETAILED DESCRIPTION OF THE INVENTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to that this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The disclosure of all patents, patent applications (and any patents that issue thereon, as well as any corresponding published foreign patent applications), GenBank and other accession numbers and associated data, and publications mentioned throughout this description are hereby incorporated by reference herein. It is expressly not admitted, however, that any of the documents incorporated by reference herein teach or disclose the present invention.

The present invention may be understood more readily by reference to the following detailed description of the embodiments of the invention and the Examples included herein. However, before the present methods, compounds and compositions are disclosed and described, it is to be understood that this invention is not limited to specific methods, specific nucleic acids, specific polypeptides, specific cell types, specific host cells or specific conditions, etc., as such may, of course, vary, and the numerous modifications and variations therein will be apparent to those skilled in the art. It is also to be understood that the terminology used herein is for the purpose of describing specific embodiments only and is not intended to be limiting.

As described in the Examples below, we disclose herein compositions in the form of nucleotide aptamerss that are capable of binding PrP, and in some embodiments, differentially binding PrP isoforms. Also disclosed are methods for identifying PrP in a sample, and in some embodiments, either selectively removing PrP or PrP isoforms from a sample, or inactivating them within a sample.

As used herein, the term “Aptamer” refers to a nucleic acid that binds to another molecule (“target,” as described below). This binding interaction does not encompass standard nucleic acid/nucleic acid hydrogen bond formation exemplified by Watson-Crick base pair formation (e.g., A binds to U or T and G binds to C), but encompasses all other types of non-covalent (or in some cases covalent) binding. Non-limiting examples of non-covalent binding include hydrogen bond formation, electrostatic interaction, Van der Waals interaction and hydrophobic interaction. An aptamer may bind to another molecule by any or all of these types of interaction, or in some cases by covalent interaction. Covalent binding of an aptamer to another molecule may occur where the aptamer or target molecule contains a chemically reactive or photoreactive moiety. The term “aptamer” or “specifically binding nucleic acid” refers to a nucleic acid that is capable of forming a complex with an intended target substance. “Target-specific” means that the aptamer binds to a target analyte with a much higher degree of affinity than it binds to contaminating materials. According to the instant invention, PrP, variants and isoforms thereof are targets. Methods of constructing and determining the binding characteristics of aptamers are well known in the art. For example, such techniques are described in Lorsch and Szostak (1996) and in U.S. Pat. Nos. 5,582,981, 5,595,877 and 5,637,459, each incorporated herein by reference. Aptamers may be prepared by any known method, including synthetic, recombinant, and purification methods, and may be used alone or in combination with other aptamers specific for the same target. Further, the term “aptamer” specifically includes “secondary aptamers” containing a consensus sequence derived from comparing two or more known aptamers that bind to a given target. In general, aptamers of a minimum of approximately 10 to 40 nucleotides in length or more, are used to effect specific binding. Although the nucleic acid ligands described herein are single-stranded or double-stranded, it is contemplated that aptamers may sometimes assume triple-stranded or quadruple-stranded structures.

The aptamers contain the sequence that confers binding specificity, but may be extended with flanking regions and otherwise derivatized. In some embodiments of the invention, aptamer binding sites will be flanked by known, amplifiable sequences, facilitating the amplification of the nucleic acid ligands by PCR or other amplification techniques. In a further embodiment, the flanking sequence may comprise a specific sequence that preferentially recognizes or binds a moiety to enhance the immobilization of the aptamer to a substrate. The flanking sequences may also contain other convenient features, such as restriction sites. These primer hybridization regions generally contain 10 to 30, 15 to 25, and in some embodiments 18 to 30, bases of known sequence. In yet other embodiments, these primer or overhang regions may comprise randomly from 4 to 10 bases. Both the randomized portion and the primer hybridization regions of the initial oligomer population may be constructed using conventional solid phase techniques. Such techniques are well known in the art, such methods being described, for example, in Froehler, et al., (1986a, 1986b, 1988, 1987). Nucleic acid ligands may also be synthesized using solution phase methods such as triester synthesis, known in the art. For synthesis of the randomized regions, mixtures of nucleotides at the positions where randomization is desired are added during synthesis. Any degree of randomization may be employed. Some positions may be randomized by mixtures of only two or three bases rather than the conventional four. Randomized positions may alternate with those which have been specified. Indeed, it is helpful if some portions of the candidate randomized sequence are in fact known.

The aptamerss may be isolated, sequenced, and/or amplified or synthesized as conventional DNA or RNA molecules. Alternatively, nucleic acid ligands of interest may comprise modified oligomers. Any of the hydroxyl groups ordinarily present in nucleic acid ligands may be replaced by phosphonate groups, phosphate groups, protected by a standard protecting group, or activated to prepare additional linkages to other nucleotides, or may be conjugated to solid supports. The 5′ terminal OH is conventionally free but may be phosphorylated. Hydroxyl group substituents at the 3′ terminus may also be phosphorylated. The hydroxyls may be derivatized by standard protecting groups. One or more phosphodiester linkages may be replaced by alternative linking groups. These alternative linking groups include, exemplary embodiments wherein P(O)O is replaced by P(O)S, P(O)NR.sub.2, P(O)R, P(O)OR′, CO, or CNR.sub.2, wherein R is H or alkyl (1-20 C) and R′ is alkyl (1-20 C); in addition, this group may be attached to adjacent nucleotides through O or S. Not all linkages in an oligomer need to be identical.

The SELEX method involves selection from a mixture of candidate nucleic acid ligands and step-wise iterations of binding, partitioning and amplification, using the same general selection scheme, to achieve virtually any desired criterion of binding affinity and selectivity. Starting from a mixture of nucleic acid ligands comprising a segment of randomized sequence, the method includes the following steps. Contacting the mixture with the target under conditions favorable for binding. Partitioning unbound nucleic acid ligands from those nucleic acid ligands that have bound specifically to target analyte. Dissociating the nucleic acid ligand-analyte complexes. Amplifying the nucleic acid ligands dissociated from the nucleic acid ligand-analyte complexes to yield mixture of nucleic acid ligands that preferentially bind to the analyte. Reiterating the steps of binding, partitioning, dissociating and amplifying through as many cycles as desired to yield highly specific, nucleic acid ligands that bind with high affinity to the target analyte.

In the SELEX process, a candidate mixture of nucleic acid ligands of differing sequence is prepared. The candidate mixture generally includes regions of fixed sequences (i.e., each of the nucleic acid ligands contains the same sequences) and regions of randomized sequences. The fixed sequence regions are selected to: (a) assist in the amplification steps; (b) mimic a sequence known to bind to the target; or (c) promote the formation of a given structural arrangement of the nucleic acid ligands. The randomized sequences may be totally randomized (i.e., the probability of finding a given base at any position being one in four) or only partially randomized (i.e., the probability of finding a given base at any location can be any level between 0 and 100 percent).

The candidate mixture is contacted with the selected analyte under conditions favorable for binding of analyte to nucleic acid ligand. The interaction between the target and the nucleic acid ligands can be considered as forming nucleic acid ligand-target pairs with those nucleic acid ligands having the highest affinity for the analyte.

The nucleic acid ligands with the highest affinity for the analyte are partitioned from those nucleic acid ligands with lesser affinity. Because only a small number of sequences (possibly only one molecule of nucleic acid ligand) corresponding to the highest affinity nucleic acid ligands exist in the mixture, it is generally desirable to set the partitioning criteria so that a significant amount of nucleic acid ligands in the mixture (approximately 5-50%) are retained during partitioning. Those nucleic acid ligands selected during partitioning as having higher affinity for the target are amplified to create a new candidate mixture that is enriched in higher affinity nucleic acid ligands. By repeating the partitioning and amplifying steps, each round of candidate mixture contains fewer and fewer weakly binding sequences. The average degree of affinity of the nucleic acid ligands to the target will generally increase with each cycle. The SELEX process can ultimately yield a mixture containing one or a small number of nucleic acid ligands having the highest affinity for the target analyte. Nucleic acid ligands produced for SELEX may be generated on a commercially available DNA synthesizer. The random region is produced by mixing equimolar amounts of each nitrogenous base (A, C, G, and T) at each position to create a large number of permutations (i.e., 4ⁿ, where “n” is the oligo chain length) in a very short segment. Thus a randomized 40 mer (40 bases long) would consist of 4⁴⁰ or maximally 10²⁴ different nucleic acid ligands. This provides dramatically more possibilities to find high affinity nucleic acid ligands when compared to the 10⁹ to 10¹¹ variants of murine antibodies produced by a single mouse. The random region is flanked by two short Polymerase Chain Reaction (PCR) primer regions to enable amplification of the small subset of nucleic acid ligands that bind tightly to the target analyte.

As used herein, the term “PrP^{c}” refers to the cellular isoform of the prion protein as well as fragments and derivatives thereof irrespective of the source organism. The term PrP^{Sc} refers to the isoform of the prion protein associated with various transmissible spongiform encephalopathies, fragments of this prion protein isoform, proteins of the various Scrapie strains including those adapted to hamster, mouse or other vertebrates, and derivatives of the prion protein isoform PrP^{Sc}. As used in connection with PrP or prion, the term “derivatives” includes chemically modified versions of the prion protein isoforms PrP^{c} and PrP^{Sc} as well as mutants of these proteins, namely proteins which differ from the naturally occurring prion protein isoforms at one or more positions in the amino acid sequence, as well as proteins that show deletions or insertions in comparison to the naturally occurring prion protein isoforms. Such mutants can be produced by recombinant DNA technology or can be naturally occurring mutants, such as variants that may be found within or among various animal species. The term derivatives also embraces proteins which contain modified amino acids or which are modified by glycosylation, phosphorylation and the like.

In some embodiments according to the invention, the nucleic acid molecules may be modified at one or more positions in order to increase their stability and/or to alter their biochemical and/or biophysical properties. In some embodiments according to the invention, the compositions disclosed herein may include pharmaceutically acceptable carriers. These compositions may be useful for the therapy of transmissible spongiform encephalopathies such as those listed above. It may be possible, for example, to suppress the conversion of the non-disease causing isoform PrP^{c} into the prion associated isoform PrP^{Sc}, such as by applying nucleic acid molecules which specifically bind to PrP^{c}.

The present invention also provides in some embodiments diagnostic compositions comprising nucleic acid molecules according to the invention. Such compositions may contain additives commonly used for diagnostic purposes. The nucleic acid molecules and the diagnostic compositions according to the invention can be used in methods for the diagnosis of transmissible spongiform encephalopathies. Such a method comprises, for example, the incubation of a sample taken from a body with at least one kind of nucleic acid molecules according to the invention and the subsequent determination of the interaction of the nucleic acid molecules with the isoforms PrP^{c} and PrP^{Sc} of a prion protein. According to some embodiments, at least one nucleic acid composition described herein can be used to quantitatively determine the amount of at least one isoform of a prion protein in a sample, and in some embodiments to determine the absolute and/or relative amount of one or more isoforms in a sample.

According to the various embodiments wherein the compositions herein are used to test a sample, the sample may be obtained from various organs, preferably from tissue, for example, from brain, tonsils, ileum, cortex, dura mater, Purkinje cells, lymphnodes, nerve cells, spleen, muscle cells, placenta, pancreas, eyes, backbone marrow or peyer'sche plaques, or from a body fluid, such as blood, cerebrospinal fluid, milk or semen.

### EXAMPLES

### Example 1

### Preparation of Aptamers that Distinguish Between Prion Isoforms

DNA aptamers were selected against rhuPrP via the SELEX procedure, using lateral flow chromatography. We generated a panel of DNA aptamers that bind to recombinant PrPC and immunoprecipitated mammalian PrPC derived from a variety of animal species. Further, these DNA aptamers did not bind to PrPSc and other neuroproteins.

Materials and Methods

Materials for SELEX. An aptamer library was synthesized (Integrated DNA technology, Inc., Coralville, Iowa) that consisted of a randomized 40-mer DNA sequence flanked by two known 28-mer primer-binding sites:

(59-TTTGGTCCTTGTCTTATGTCCAGAATGC-N40-ATTTCTCCTACTGGGATAGGTGGATTAT-39: where N40 represents 40 random nucleotides with equimolar A, C, G, and T)

The same manufacturer was used to synthesize all primers and aptamers applied in this study. The rhuPrPC fragment consisting of amino acid residues 23-231 (rhuPrPC23-231) served as the target protein. A device for lateral flow chromatography (6 mm 3 65 mm) consisting of a nitrocellulose (NC) membrane immobilized on a polymer support with an aptamer releasing pad at one end and a wicking pad at the other end, was used as the solid-phase support for the SELEX procedures.

SELEX and Synthesis of Selected Aptamers. The aptamer library was enriched for the selection of specific aptamer candidates against rhuPrPC23-231 by SELEX enrichment, using a lateral flow chromatography device. Sixty nanograms of rhuPrPC23-231 was deposited as a line at the center of the NC membrane and immobilized by air-drying. The NC membrane was blocked with 1% bovine serum albumin (BSA) in phosphate-buffered saline (PBS) containing 0.05% Tween-20 (PBST). The aptamer library was diluted in PBST containing 1% BSA and applied to the releasing pad. After DNA molecules passed through the NC membrane, the solid phase was washed six times with a high-stringency washing buffer (2.2 g Ncyclohexyl-3-aminopropanesulfonic acid, 11.7 g potassium thiocyanate, 0.2 g NaN3, 21.3 g Triton X-100, 40 ml of 253 PBS, and 950 ml of dH2O; pH adjusted to 7.6 with 10 N NaOH; dH2O added to bring volume to 1000 ml). The region of the NC membrane coated with rhuPrPC23-231, where the high-affinity aptamers were expected to bind, served as a template for polymerase chain reaction (PCR). Amplification was carried out with a set of primers, of which, one (59-ATAATCCACCTATCCCAGTAGGAGAAAT-39) was biotinylated at the 59 end to enable easy removal of the reverse complement orientation of the original library using streptavidin-coated magnetic beads (Promega Co., Madison, Wis.). Unbiotinylated strands (representing the orientation of the original library) were reused for the subsequent rounds of SELEX. Six iterations of SELEX were performed. Binding specificity and affinity of the sixth aptamer pool were investigated by chemiluminescent dot blot and gel shift analyses. The candidates in the selected aptamer pool after the sixth SELEX were cloned into TA vectors (TOPO II; Invitrogen Co., Carlsbad, Calif.), and 50 clones were sequenced. Based on the frequency of common sequences found among 50 clones and the theoretical secondary structures obtained using thermodynamics and mathematical-modeling procedures (17, 18), eight selected sequences were synthesized for specificity and sensitivity evaluation. The synthesized aptamers were 59 biotinylated to enable detection. The end point concentrations at which aptamers bound to rhuPrPC23-231 were measured using an enzyme-linked immunosorbent assay (ELISA) format; whereby 59 biotinylated aptamers were incubated in rhuPrPC23-231- or rhu90-231 (rhuPrPC fragment consisting of amino acid residues 90-231)-coated 96-well microtiter plates, followed by detection with neutravidin horseradish peroxidase (HRP) conjugate as described in the section entitled, “End Point Concentration at Which Aptamers Bound to rhuPrPC23-231.”

Construction of Truncated Aptamers. Sequences of aptamers derived by SELEX are presented in Table 1. Short aptamers consisting of the randomized region alone in sense and antisense orientations with and without flanking overhangs were constructed and biotinylated at the 59 end. We chose the most frequently identified aptamer (designated as 3-10), which also showed a greater binding ability to PrPC. This aptamer represented 32% of the sequences identified among 50 clones sequenced after the sixth round of SELEX procedure.

Immunoprecipitation of Mammalian PrPs. PrPC was purified and concentrated from brain tissue from apparently healthy animals (sheep, pigs, white tailed deer, and calves). One part of brain tissue was homogenized in nine parts of lysis buffer (10 mM Tris, 150 mM NaCl, 1% Nonidet P-40, 0.5% deoxycholate, and 5 mM EDTA, pH 8.0) containing 1 mM phenylmethylsulfonyl fluoride. The brain homogenate was centrifuged at 11,700 g for 10 mins, and the supernatant was stored in aliquots at −808 C before use. The monoclonal antibody (mAb), FH11 (TSE Resource Center, Institute for Animal Health, Berkshire, UK), was covalently immobilized onto an agarose gel using the Seize Primary Immunoprecipitation Kit (Pierce, Rockford, Ill.). The brain supernatant was added to the antibody-coupled gel, and immunoprecipitation was performed as suggested by the manufacturer. The eluted PrP fractions were dialyzed against PBS buffer (pH 7.5) and concentrated using centrifugal filter units (Centricon Centrifugal Filter Units, MWCO 10000; Millipore, Billerica, Miss.). The concentration of purified PrP was measured by bicinchonic acid protein assay (Pierce). The purified PrP was stored at −808 C before use. A protein profile of purified PrP was generated by sodium dodecyl sulfate (SDS)-polyacrylamide gel electrophoresis (PAGE) followed by Western blot using a mAb, BG4 (TSE Resource Center, Institute for Animal Health).

Dot Blot Analysis. RhuPrPC23-231, rhuPrPC90-231, casein (used as a nonspecific protein), and biotinylated primer alone (as a positive control for the assay) were immobilized as dots on an NC membrane by air-drying for proteins and by UV-linking for nucleotides. The membrane was blocked with 1% BSA in PBST and incubated with heat-denatured biotinylated aptamers from the sixth SELEX enrichment. The membrane was washed three times with PBST and incubated with streptavidin-alkaline phosphate conjugate (Promega). After three washes with PBST, the membrane was equilibrated with a detection buffer (0.1 M Tris-HCl and 0.1 M NaCl, pH 9.5). A chemiluminescent substrate (CDP-star, ready-to-use; Roche, Basel, Switzerland) was added to the membrane and the signal was detected using a ChemiImager 5500 (Alpha Innotech Corporation, San Leandro, Calif.), with a chemiluminescent filter, for 5 to 15 mins.

Gel Shift Analysis. Synthesized aptamers (10-10 M to 10-12 M) or heat-denatured amplicons of the sixth SELEX aptamer pool were incubated with 1 lag rhuPrPC23-231 for 30 mins at room temperature. The mixture was resolved by 13 0Tris-borate-EDTA (TBE)-buffered native PAGE. When amplicons of the sixth SELEX aptamer pool were used, the aptamers were directly visualized by ethidium bromide staining. When biotinylated aptamers were used, the aptamers were transferred onto a positively charged nylon membrane (Schleicher & Schuell Inc., Keene, N.H.) and detected by chemiluminescence techniques, as described above in the Dot Blot Analysis section.

3SDS-PAGE and Detection with Aptamers (South-Western Blot Analysis). Recombinant huPrPC23-231 was separated by SDS-PAGE (19) and transferred to an NC membrane by electroblotting at 60 V for 2 hrs. The NC membrane was blocked with 0.2% Blocking Reagent (Roche Diagnostics Co., Indianapolis, Ind.) in PBST, followed by incubation with 10-10 M selected aptamers for 3 hrs, Binding was detected using chemiluminescence methods as described above in the Dot Blot Analysis section.

End Point Concentrations at Which Aptamers Bound to rhuPrPC23-231. Microtiter plates were coated with 100 ng rhuPrPC23-231 in carbonate buffer (15 mM Na2CO3 and 35 mM NaHCO3, pH 9.6) overnight at 48 C. The plates were washed three times with PBST and blocked with 1% BSA in PBST at 378 C for 2 hrs. Synthesized aptamers were diluted in PBST containing 1% BSA at a final concentration of 1 mM, and serially (10-fold) diluted in the microtiter plates. PBS was used as a control. The plates were incubated at room temperature for 3 hrs, followed by three washes with PBST. The biotin label of the bound aptamers was detected by neutravidin-HRP conjugate (Pierce) diluted 1:1000 in PBST containing 1% BSA. A substrate (3,39,5,59-tetramethylbenzidine; Sigma-Aldrich, St. Louis, Mo.) was added to the plates, and the reaction was stopped by the addition of 5% HCl. The optical density was determined at 450 nm. The end point was defined as the dilution at which the optical density of sample wells exceeded the mean optical density of 12 control wells plus 3 standard deviations. The assay was repeated six times.

Cell Lines. Scrapie-infected mouse neuroblastoma cell line (ScN2a) was purchased from InPro Biotechnology, Inc. (South San Francisco, Calif.). Mouse PrP-null (PsFF)1 and PrPC-overexpressing (Mo3F4) lines used in cell blots were constructed in the laboratory of S.A.P. (20).2

Cell Blot. Cell blot analyses were performed using standard procedures, as described (21). In brief, cells were grown in Dulbecco's modified Eagle's medium (DMEM; Quality Biological, Inc., Gaithersburg, Md.) supplemented with 4 mM L-glutamine, 10% fetal calf serum, and 100 U/ml penicillin/streptomycin on plastic cover slips placed in the wells of a 24-well plate in 5% CO2 at 378 C for 4 days. Cells were blotted onto an NC membrane by applying firm pressure for 30 secs. The NC membrane was air-dried and incubated in a lysis buffer (0.5% deoxycholate, 0.5% Triton X-100, 150 mM NaCl, and 10 mM Tris-HCl, pH 7.5) with or without 5 gg/ml PK for 1.5 hrs at 378 C. The NC membrane was washed in distilled water and incubated for 20 mins with 5 mM phenylmethylsulfonyl fluoride at room temperature. The membrane was immersed in denaturing buffer (3 M guanidine isothiocyanate and 10 mM Tris-HCl, pH 8.0) for 10 mins, washed three times in water, and blocked in Tris-buffered saline (TBS) containing 0.1% Tween-20 (TBS-T) and 5% nonfat dried milk for 2 hrs. When the assay was performed against native PrP, the NC membrane was blocked in 5% nonfat dried milk without treatment with denaturing buffer. After blocking, the membrane was incubated with the mAbs FH11 (1:5000) or GE8 (1:5000; TSE Resource Center, Institute for Animal Health), or with aptamers (108 M). As a positive control, anti-14-3-3 mAb (1:5000; Upstate Biotechnology, Lake Placid, N.Y.) was used to detect neuroblastoma cells on a NC membrane. mAbs and aptamers were detected with anti-mouse IgG-HRP conjugate (1:10,000) and neutravidin-HRP conjugate (1:1000; Pierce), respectively. A chemiluminescent substrate (ECL Plus Western Blotting Detection Reagents; Amersham Biosciences Inc., Piscataway, N.J.) was added, and signal was captured using the ChemiImager 5500 (Alpha Innotech Corporation), with a chemiluminescent filter, for 5 to 15 mins.

Results

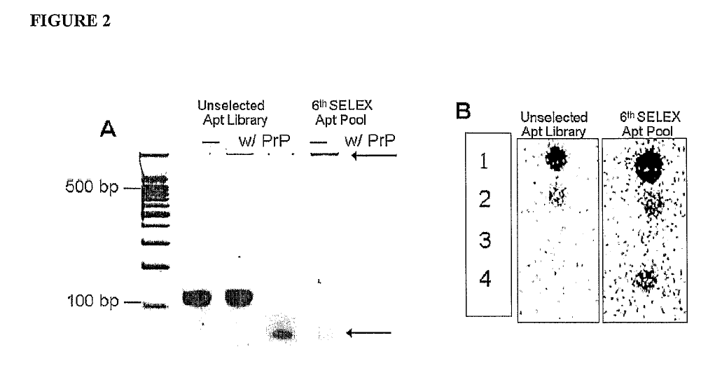

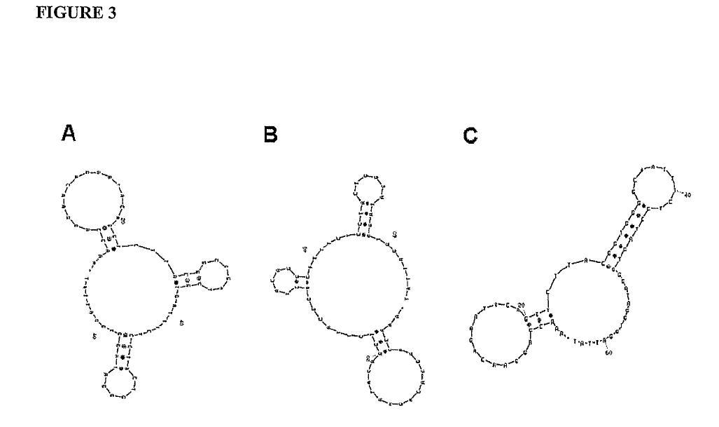

Selection of DNA Aptamers Against rhuPrP. After six rounds of SELEX procedures, the selected aptamer pool demonstrated higher affinity to rhuPrPC23-231 than that of the original aptamer library by gel shift (FIG. 2A) and dot blot analyses (FIG. 2B), indicating that the SELEX procedure selectively enriched for aptamers with higher affinity to rhuPrPC23-231. Therefore, the sixth-round aptamer pool was cloned and 50 clones were nucleotide sequenced. We initiated the SELEX with an aptamer library containing 40 randomized nucleotides, however, as a result of six rounds of the SELEX procedure, the randomized region of selected aptamers became 8-10 bp in length. Three of the selected candidates, designated 3-10, 1-2, and 1-7, represented 32%, 8%, and 5% of the sequences, respectively. Sequences of these aptamers are shown in Table 1. Among all sequences obtained, based on the frequency of occurrence in the 50 sequences and theoretical structures (17), eight sequences were selected for synthesis (aptamer SSAP1-2, SSAP1-4, SSAP1-6, SSAP1-9, SSAP1-13, SSAP3-10, SSAP3-24, and SSAP3-59; Table 1) and PrP-binding studies. Representative structures (17) of the aptamers (FIG. 3) indicated that the selected candidate sequences participated in the formation of stem-loop-like structures.

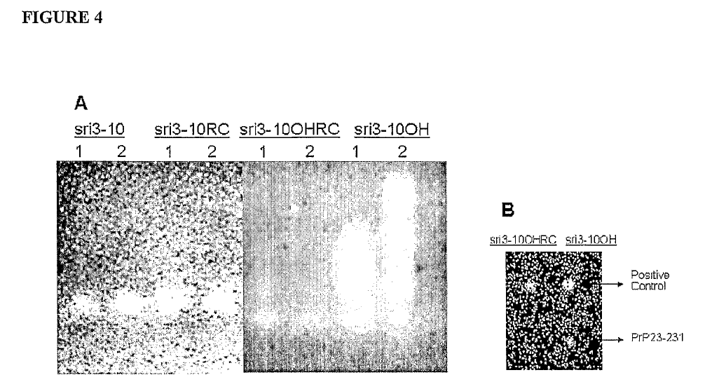

Aptamer-rhu PrP-Bind ing Studies Using Denaturing and Nondenaturing Conditions. To investigate the role of secondary structures of the randomized region in PrP binding, short aptamers derived from SSAP3-10 were synthesized and analyzed for their binding to rhuPrPC23-231. A short aptamer designated sri3-10OH that consisted of the aptamer 3-10 randomized region in the correct orientation with trimer and tetramer flanking overhangs bound to rhuPrPC23-231, but other short aptamers (randomized sequence alone, reverse complement of randomized sequence, and the reverse complement of randomized sequence with 3- and 4-bp overhangs) did not demonstrate binding by gel-shift analyses (FIGS. 4A and B). A native gel electrophoretic pattern of sri3-10OH showed multiple bands, suggesting the presence of several secondary structures (FIG. 4A).

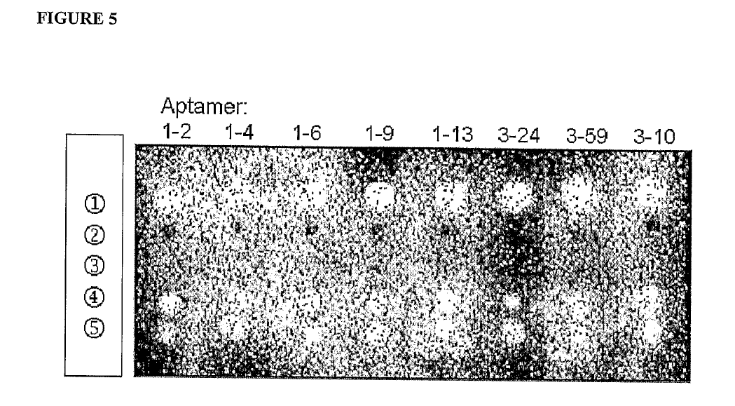

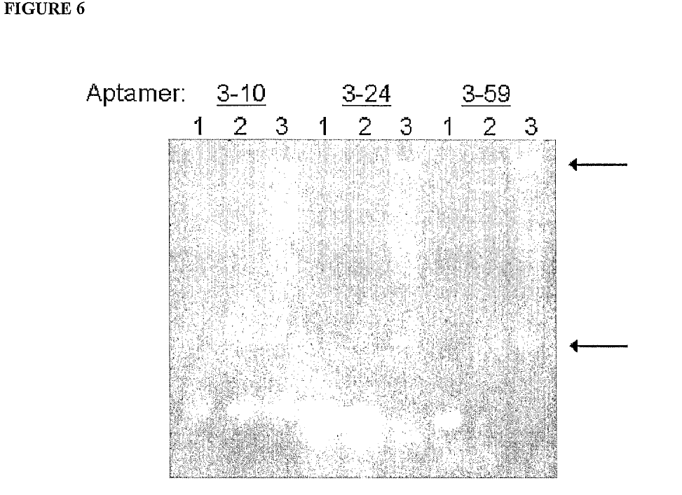

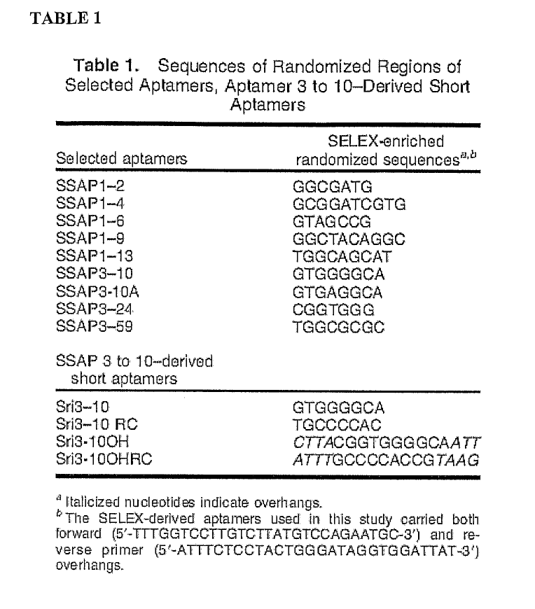

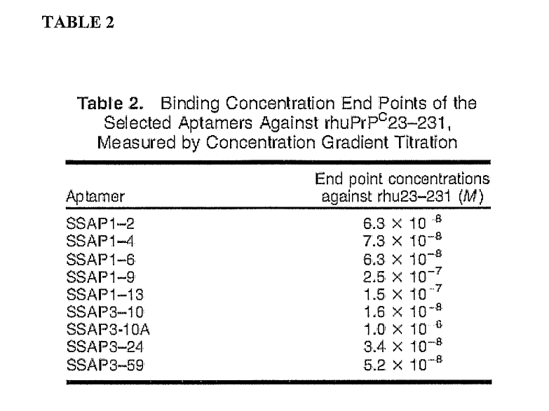

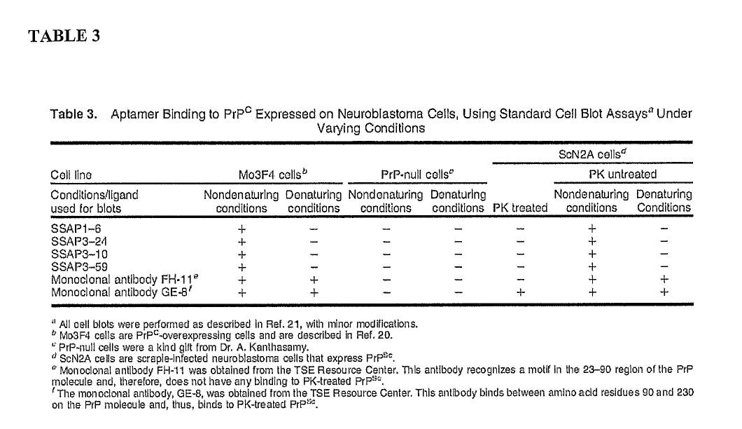

All of the selected aptamers showed affinity to rhuPrPC23-231 by gel shift and dot blot analyses (FIG. 5). The dot blot analysis indicated that the aptamers bound to rhuPrPC23-231, but not to rhuPrPC90-231 at 10-10 M. Selected aptamer candidates also detected denatured rhuPrPC23-231, but not rhuPrPC90-231 in South-Western blots (data not shown).

Binding concentration end points of the selected aptamers to rhuPrPC23-231 measured by a dilution-to extinction titration method ranged from 10-7 to 108 M (Table 2). A single base change of the randomized region of aptamer 3-10 (G to A, designated as 3-10A, Table 1) increased the end point of modified aptamer by 2 logs from that of the original aptamer 3-10 (Table 2). Titration to extinction experiments using rhu90-231 indicated that two aptamers bound at 10-8 M concentrations. The aptamer 3-10 bound to rhu90-231 at concentrations of 10-6 M and greater.

Selected DNA Aptamers Bind to Mammalian PrPs. Using our panel of DNA aptamers that reproducibly bound to a recombinant PrP at nanomolar concentrations, we investigated their binding abilities to mammalian PrPs enriched by immunoprecipitation of brain extracts and PrPC expressed in cell cultures.

We used immunoprecipitation to purify (SDS-PAGE and Western blot data not shown) and concentrate PrPs from brain homogenates of healthy sheep, calves, piglets, and deer, with a final concentration of approximately 0.5 mg/ml. All eight selected aptamers bound to immunoprecipitated sheep PrP by dot blot analyses (FIG. 5) and gel shift (representative data for three aptamers are shown in FIG. 6). Although the dot blot analysis was not quantitative, selected aptamers seemed to bind to immunoprecipitated sheep, bovine, porcine, and deer PrPs with varying affinities (FIG. 7).

Selected aptamers bound to mammalian PrPC expressed in Mo3F4 cells as shown (Table 3) when the cells were immobilized on NC membranes. Neither the selected (SELEX derived) nor the simulated aptamers generated any signal against PrP-null cells (Table 3). Anti-PrP mAbs FH11 (Table 3) and GE8 (Table 3) also did not generate a signal against PrP-null cells. We used 14-3-3y, an intracellular neuroprotein, as a positive control for cell blot analysis, because it is a neuronal protein that is abundant in most areas of central nervous system (22). Anti-14-3-3 mAb gave positive signals in both PrP-null (Table 3) and ScN2a cells (Table 3). The selected aptamers did not bind to 14-3-3 (Table 3) nor to other neuroproteins expressed by PrP-null cells by gel shift, dot blot, and South-Western blot analyses (data not shown). Selected aptamers detected PK untreated PrP expressed by ScN2a cells (Table 3). The epitope of anti-PrP mAb GE8 is located in the C-terminus of PrP. mAb GE8 detected PrP from PK-treated ScN2a cells (Table 3), indicating that the ScN2a cells expressed PrPSc. In contrast, mAb FH11 did not generate any signal against PK-treated ScN2a cells in the N-terminus of PrP, because the epitope of FH11 is expected to be degraded (Table 3). Aptamers did not bind to the PK-digested PrP fragments in ScN2a cells (Table 3). PK treatment digested the N-terminus of PrPSc, therefore, the remaining products were considered to be mostly PrPSc fragments containing amino acid residues 90-231 (1). This finding concurred with our observation that aptamers bound to recombinant PrP23-231 but not to recombinant PrP90-231.

Discussion

This study was undertaken to develop ligands that could potentially differentiate normal and abnormal prion isoforms. Toward this end, we undertook a well-established SELEX protocol to generate a panel of DNA aptamers against PrPC. We tested their abilities to bind to several segments of PrP as well as normal and abnormal prion isoforms to evaluate their usefulness in diagnostics of prion disease. Interest in the pathobiology and epidemiology of human and animal prion diseases has recently accelerated for several reasons. First, the mounting experimental evidence has generated great interest in what seems to be a protein-initiated mechanism of disease (23-26). Second, the demonstration that prions are responsible for BSE (27-30), which has infected large numbers of cattle in Great Britain, the recent report of a case of BSE in the United States, and the presence of chronic wasting disease (CWD) in feral and captive deer populations have increased the concern that animal-to-human transmission of prion disease poses a substantial threat to the human race and its food chain; clearly much more effort is needed to prevent this possible epidemic and has lent a new urgency to the quest for accurate diagnostic tools and efficacious therapeutic tools.

PrPC is a sialoglycoprotein bound to the cell surface through a glycosyl phosphatidyl-inositol anchor. The infectious isoforms or PrPSc differ from PrPC in that they are insoluble in nonionic detergents or chaotropic agents and are partially PK resistant (31). Indeed, these characteristics of prions are applied in currently available diagnostics to identify the presence of an infectious form of the prion protein for confirmatory diagnosis in postmortem tissue. Thus, the development of diagnostic tools that are more sensitive in addition to the identification and manufacture of optimal ligands (such as antibodies, receptors, or aptamers) that are able to differentiate prion isoforms will be very useful in generating safe foods and pharmaceuticals. These ligands will also become an integral part of the diagnostic armamentarium of prion disease and prion detection.

Aptamers Enriched by SELEX Bind to Both Recombinant and Mammalian PrPC and Not to PrPSc. SELEX-derived aptamers detected rhuPrPC23-231 when PrP was presented in its native form. Although the aptamers were selected against, and reacted with, a recombinant full-length prion fragment, they also showed affinity to mammalian PrPC concentrated from brain homogenates and cultured cells. Aptamers, like some currently available antibodies, bind to PrPC despite the presence of large glycans in mammalian PrP at amino acid residues N181 and N197 (32). Because PrP is a highly conserved protein among animals and humans (1), it may be a challenge to generate antibodies that differentiate PrPs from different species. Our results demonstrate that the selected aptamers detect immunoprecipitated PrP from sheep, calf, piglet, and white-tailed deer, suggesting that species- and isoform-specific DNA aptamers could be selected. These studies are currently underway in our laboratory.

The binding sites of six aptamers identified in this study are located between amino acid residues 23 and 89 of PrP. This finding is congruent with previous studies with RNA aptamers selected against PrPs, which showed that an RNA aptamer selected against recombinant hamster PrP23-231 bound to the PrP fragment containing amino acid residues 23-52 (13). In the presence of a mAb directed against amino acid residues 37-53 of PrP, the RNA aptamer retained its affinity for PrP23-231, indicating that the RNA aptamer interacted with PrP through amino acid residues 23-36 of PrP (13). Another study using rhuPrP to characterize RNA aptamer binding suggested that PrP possessed two RNA binding sites: one was found in the N-terminus between amino acid residue 23 and 90 and the other was in the C-terminal core structure of PrP (15). Although binding of these RNA aptamers to the in vitro-derived 0-form of the prion was shown, neither its reactivity to mammalian prions nor the specificity to PrPSc in a background of large amounts of nonspecific host proteins were shown. In contrast, DNA aptamers identified in the current study were able to bind to prions derived from a variety of host species.

Our data indicated that there was good affinity between PrPC and the selected aptamers, and that the binding was specific to PrPC in its native form, as demonstrated by the lack of reactivity to other neuroproteins expressed by PrPnull cells. The selected aptamers seem to recognize the N-terminus of PrP, where PrP is rather flexible and lacks defined secondary structures (33). Thus, the data strongly suggest that the selected aptamers were PrPC conformation specific. These findings parallel those reported by Sayer et al. (16) on PrPSc specific aptamers and indicate that aptamers could be applied to the differentiation of prion conformations, Taken together, the panel of selected aptamers specifically bound to a PrPC conformation and not to PrPSc or to other neuroproteins.

Studies on Aptamer-PrP Binding Kinetics Demonstrate Aptamer Sequence and Structure Specificity. Because our analyses of selected aptamers identified similarities in structures of the aptamers and suggested a sequence-structure relationship, we queried the role of their nucleotide sequences and secondary structures in binding to PrPC.

The role of secondary structures of the randomized region of selected aptamers in PrP-binding was investigated using short aptamers designed from aptamer 3-10. The data suggest that the aptamer secondary structures influenced the binding of aptamer to rhuPrPC23-231. The findings that reverse complement of sri3-OH or other sequences neither showed multiple single-strand conformations nor bound to PrPC are suggestive of sequence and structure specificity in aptamer-PrPC interactions.

A second set of studies to evaluate binding affinities and the sequence specificity of aptamer-PrP binding showed that swapping one nucleotide (GSA) within the selected region of aptamer 3-10, led to a 2 log10 drop in its binding end point to PrPC, indicating sequence specificity. In sum, these studies indicate that aptamer-PrP binding was associated with affinities comparable to those of mAbs and that the binding was aptamer-sequence specific.

That the randomized region of our library was 40-bp, but a majority of our selected aptamers was 8-10 bp in length, deserves comment. Taq polymerase, DNA polymerase from Thermus aquaticus, has domains responsible for DNA polymerase and 59 endonuclease activities (34). The endonuclease activity is structure specific and cleaves single-stranded DNA or RNA at the bifurcated end of a base-paired duplex (34). During PCR, single-stranded DNA generally forms stem-loop-like structures when heated and cooled, conditions that occur between the denaturation and annealing cycles of PCR. These structures are targets of the 59 nuclease activity of Taq polymerase for cleavage, resulting in reduced lengths of the selected aptamers. Because the DNA polymerase activity is not coupled to nuclease cleavage (34), this issue could be overcome by using the Klenow fragment, a molecule that is an N-terminal deletion mutant of Taq DNA polymerase lacking 59 nuclease activity (35). Another possible cause for the loss of nucleotides during our SELEX could have been the fact that the initial aptamer library may have contained multiple truncated products, resulting in shorter selected sequences. Nonetheless, the SELEX procedure successfully selected aptamers that specifically recognized recombinant and mammalian PrPs. Smaller randomized regions of the selected aptamers compared with the original library might have reduced its diversity. However, as Sayer et al. demonstrated (16), truncated aptamers retained their specific affinity to the recombinant target, indicating that binding ability of aptamers remains as long as its conformational specificity is conserved. This was also consistent in our truncated aptamer studies. Because the selected sequences were parts of stem-and-loop-like structures of the selected aptamers, the sequences might have been conserved during the selection because of their specific conformational binding to PrPC.

PrPC specific aptamers could serve as PrPSc-enriching reagents or as ligands in competitive transmissible spongiform encephalopathy (TSE) diagnostic assays. Because most antibodies generated to date bind to both PrPC and PrPSc, a PrPC-specific reagent, such as the aptamers we describe herein, can serve as an adjunct in current diagnostics. For example, a sample could be directly reacted with an antibody without any need for protease treatment if it has already been treated with PrPC-specific aptamers to remove all residual normal prions and, thus, simplifying the diagnostic protocol. Additionally, one could envision the application of these PrPC specific aptamers in the treatment of TSEs. In this case, these reagents could serve to bind PrPC and abrogate PrPC-PrPSc interactions, inhibiting formation of the 0-sheet-rich pathogenic isoforms.

In summary, we generated a panel of aptamers that bind to recombinant and mammalian PrPC and not to PrPSc. The PrPC specific aptamers seem to recognize a conformation and could be used in competitive or double-ligand assay formats to differentiate prion isoforms, aiding in the diagnostics of TSEs. The PrPC-specific aptamers could also be applied as therapeutic tools to deter the progression of TSEs, and some aptamers developed in these studies may find application in the future to the decontamination of blood, body fluids, foods, pharmaceuticals, and cosmetics in an automated fashion during manufacture. Because selected aptamers seemed to bind to different mammalian PrPs with varying degrees, we anticipate developing an aptamer panel that distinguishes between PrP strains and between isoforms across species. Such ligands are extremely desirable not only to detect and decontaminate pathogenic PrPs but also to accelerate molecular epidemiologic investigations of prion diseases.

### Example 2

### Counter-SELEX

Materials for SELEX—An aptamer library that consisted of a randomized 40-mer DNA sequence flanked by two known 28-mer primer binding sites (5′-TTTGGTCCTTGTCTTATGTCCAGAATGC-N40

-ATTTCTCCTACTGGGATAGGTGGATTAT-3′: where N40 represents 40 random nucleotides with equimolar A, C, G and T) was synthesized (Integrated DNA technology, Inc., Coralville, Iowa. The same manufacturer was used to synthesize all primers and aptamers applied in this study). Drowsy strain of PrPSc fragment consisting of amino acid residue 23 to 231 (Proteinase K untreated—PK−) and 90-231 (Proteinase K treated—PK+) served as target proteins. A device for lateral flow chromatography (6 mm×65 mm) consisting of a nitrocellulose (NC) membrane immobilized on a polymer support with aptamer releasing pad at one end and wicking pad at the other, was used as the solid phase support for SELEX procedures.

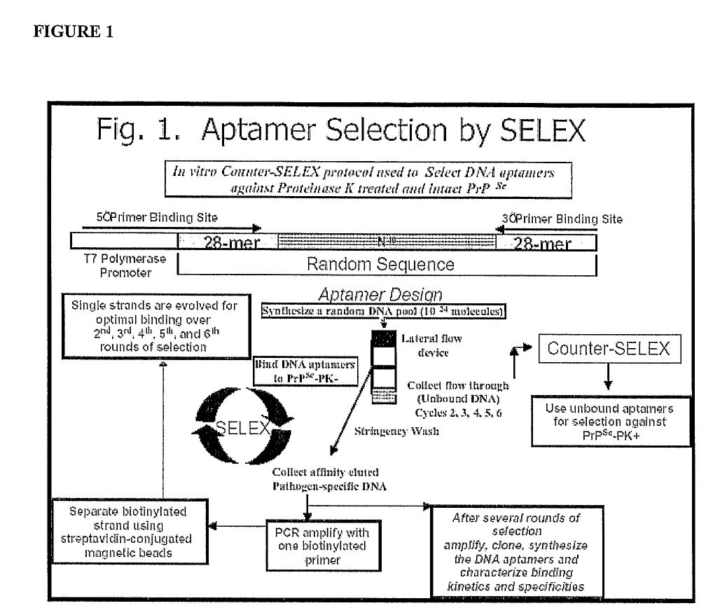

SELEX and synthesis of selected aptamers—The aptamer library was enriched for the selection of specific aptamer candidates against hamster scrapie PrPSc 23-231 (PK−) by SELEX enrichment using a lateral flow chromatography device (FIG. 1). Sixty ng of PrPSc-PK− was deposited as a line at the center of NC membrane and immobilized by air-drying. The NC membrane was blocked with 1% bovine serum albumin (BSA) in phosphate buffered saline (PBS) containing 0.05% Tween 20 (PBST). The aptamer library was diluted in PBST containing 1% BSA and applied on the releasing pad. After DNA molecules passed through PrPSc-PK− the spent library was exposed to PrPSc (PK+) coated as a second line on the NC membrane, the solid phase was washed 6 times with a high stringency-washing buffer (CAPS 2.2 g, KSCN 11.7 g, NaN3 0.2 g, Triton X-100 21.3 g, 25×PBS 40 ml, dH2O 950 ml, adjust pH to 7.6 with 10N NaOH, add dH2O to 1000 ml). The PrPSc23-231-PK− and PrPSc-90-231-PK+ coated regions of the NC membrane, where the high affinity aptamers were expected to bind, served as a template for PCR. Amplification was carried out with a set of primers of which one (5′-ATAATCCACCTATCCCAGTAGGAGAAAT-3′) was biotinylated at the 5′ end to enable facile removal of the reverse complement orientation of the original library using streptavidin coated magnetic beads (Promega Co., Madison, Wis.). Unbiotinylated strands (representing the orientation of the original library) were reutilized for the subsequent rounds of SELEX. Eight subsequent iterations of SELEX were performed independently against each molecule, respectively. Binding specificity and affinity of the 8th aptamer pool were investigated by chemiluminescent dot blot and gel shift analyses. The candidates in the selected aptamer pool after the 10th SELEX were cloned into TA vector (TOPO II, Invitrogen Co., Carlsbad, Calif.) and 50 clones for each set of PrPSc molecules (PK+ and PK−) were sequenced. Based on the frequency of common sequences found among 50 clones and the theoretical secondary structures obtained using thermodynamics and mathematical modeling procedure, 20 selected sequences against were synthesized for specificity and sensitivity evaluation. Synthesized aptamers were 5′ biotinylated to enable detection. The endpoint concentrations of which aptamers bound to rhuPrPC23-231 were measured using an ELISA format; whereby 5′ biotinylated aptamers were incubated in rhuPrPC23-231 or rhu90-231 (recombinant human PrPC fragment consisting of amino acid residue 90 to 231) coated 96-well microtiter plates followed by the detection with neutravidin horseradish peroxidase conjugate as described herein.

Results: We selected several aptamer candidates that bind to proteinase K treated or intact PrPSc. These candidates were further evaluated for specificity and binding characteristics using gel-shift and double ligand ELISA approaches.

### Example 3

### Gel-Shift Analysis of Aptamers Selected Against PrPSc

We evaluated the binding specificities of aptamer pools, after 8 rounds of SELEX, against PrPSc and PrPC molecules using gel-shift analyses.

Materials and Methods

Gel-Shift Analysis: Synthesized aptamers or heat-denatured amplicons of the 8th SELEX derived aptamer pool were incubated with rhuPrPc23-231, rhu90-231, hamster drowsy PrPSc-PK+ or hamster drowsy PrPSc-Pk− for 30 min at room temperature. The mixture was resolved by 1× Tris-Borate-EDTA (TBE) buffered native polyacrylamide gel electrophoresis. The amplicons of the 10th aptamer pool were visualized by ethidium bromide staining. The synthesized aptamers were transferred onto a positively charged nylon membrane (Schleicher & Schuell Inc., Keene, N.H.). The nylon membrane was blocked with 0.2% Blocking Reagent (Roche Diagnostics Co., Indianapolis, Ind.) in PBST followed by incubation with streptavidin-alkaline phosphate conjugate (Promega, Madison, Wis.). The nylon membrane was washed three times in PBST and equilibrated in a detection buffer. Chemiluminescent signal was obtained as described above for the dot blot analysis.

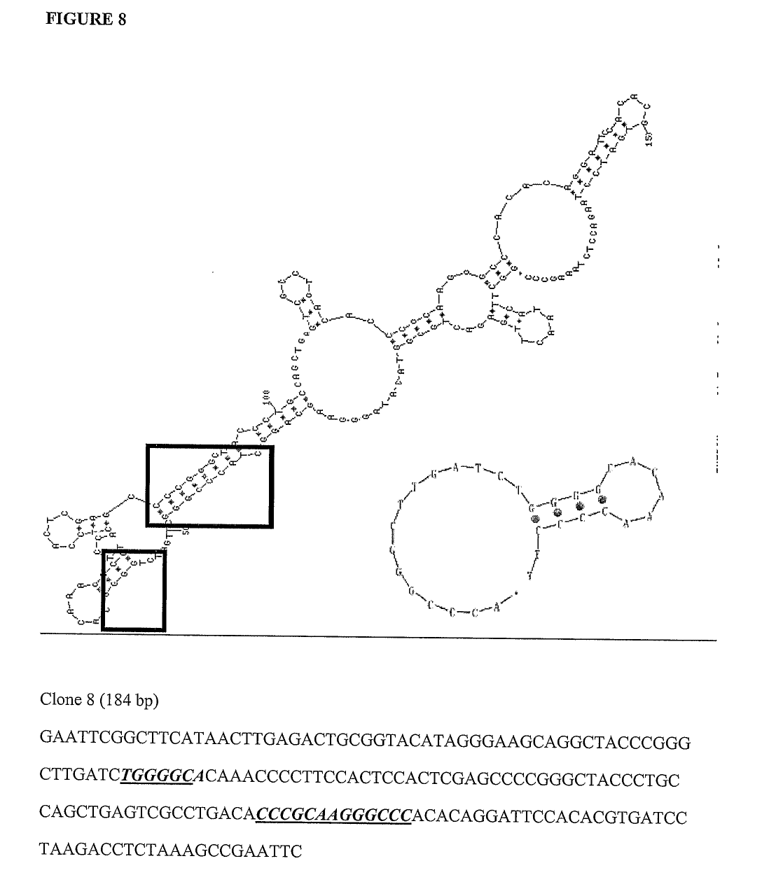

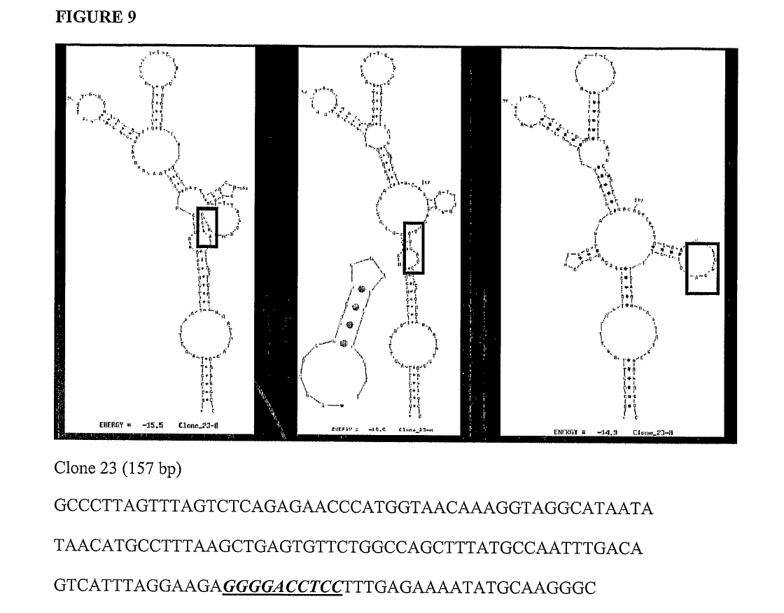

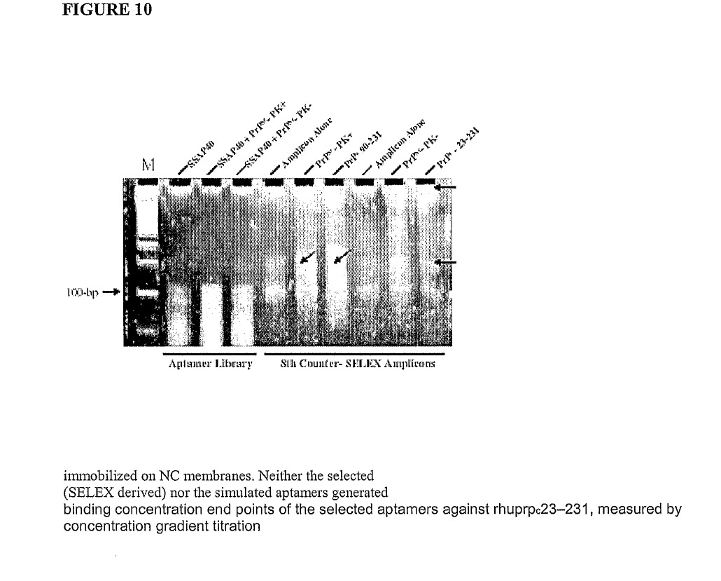

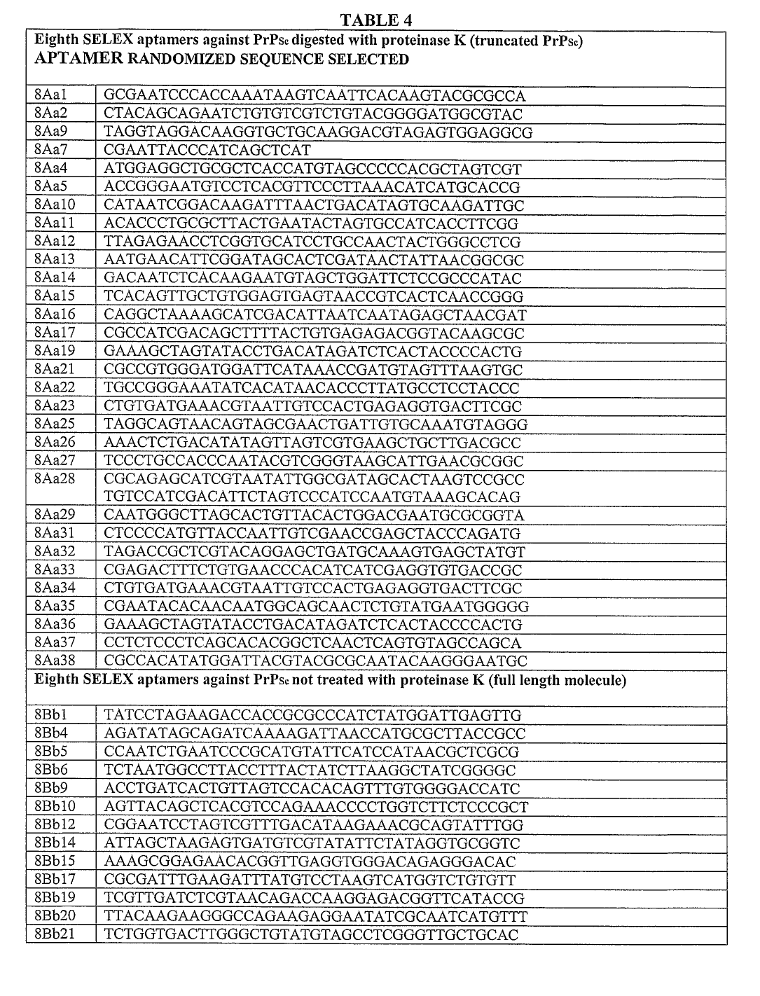

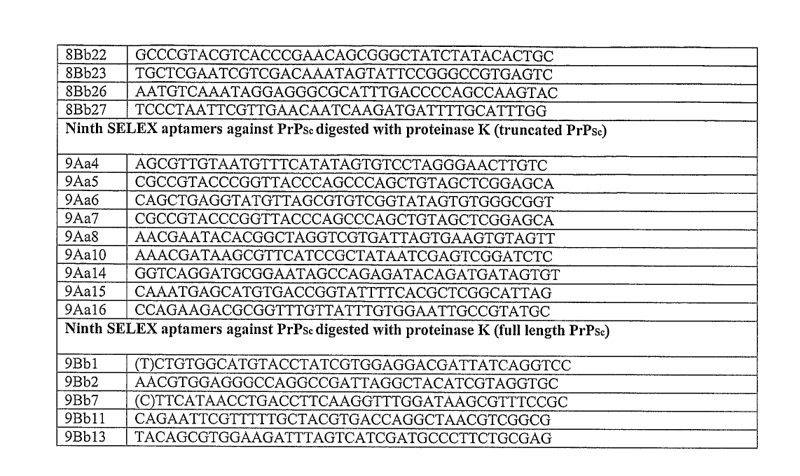

Results: The gel-shift analyses indicated that aptamer candidates were successfully selected to bind to full length and proteinase treated counterparts of PrPSc (FIG. 10). The 10th SELEX amplicons were cloned and sequenced and several aptamerss (Table 4; FIGS. 8 and 9) were identified. Good results for PrP binding have been obtained with aptamers designated 8Aa17, 9Aa10, CL 8 (FIG. 8), and CL 23 (FIG. 9). All of the shown aptamers are also candidates for discrimination between PrPSc derived from other animal species.

### Example 4

### Evaluation of PrPC Specific and PrPSc Binding Aptamers in Double Ligand Sandwich Assays to Detect Prions in Blood and Body Fluids

We used the DNA aptamers against rhuPrPc23-231 and those that bound to PrPSc in a colorimetric ELISA in combination with commercially available monoclonal antibodies 8G8 (recognizes an epitope located between amino acid residues 95 and 110) and F89 (recognizes an epitope located between amino acid residues 146 and 159). Endpoint concentration of selected aptamers bound to rhuPrPc23-231 was measured by a concentration gradient titration. Binding of aptamers to mammalian PrPs was analyzed by dilution to extinction of normal human and sheep plasma samples.

Materials and Methods

Concentration gradient titration to establish a low-end specificity for aptamer binding using colorimetric double ligand ELIAs: Microtiter plates were coated with 100 ng of either aptamers 3-10, 1-2, 3-59, and 3-10OH (described in a previous report as PrPP C specific ligands) in 1 M ammonium acetate (pH, 7.5), or 8G8 or F89 in carbonate buffer (15 mM Na CO and 35 mM NAHCO pH 9.6) over night at 4° C. The plates were washed 3 times with PBST and blocked with 1% BSA in PBST at 37° C. for 2 hours. Recombinant prion protein and/or plasma samples were serially diluted in blocking buffer. Synthesized biotinylated aptamers (specific to PrP 2 3 3, C alone or those that bound to both PrPC and PrPSc) were diluted and used in PBST containing 1% BSA at a final concentration of 1 iM. The PrP-specific antibodies (biotinylated) 8G8 or F-89 were diluted at 2.5 ig/ml and used in all analyses in combination with aptamers. All combinations of reagents with or without PrP served as negative controls. A double antibody sandwich was used as a positive control for PrPC detection. The plates were incubated at room temperature for 3 hours followed by three times wash with PBST. Biotin label of bound aptamers was detected by neutravidin-HRP conjugate (Pierce, Rockford, Ill.) diluted (1:1000) in PBST containing 1% BSA. A substrate (3,3′,5,5′-Tetramethylbenzidine, Sigma-Aldrich, St. Louis. MO) was added to the plates and the reaction was stopped by the addition of 5% HCl. The optical density was determined at 450 nm. Endpoint was defined as the dilution at which the optical density of sample wells exceeded the mean optical density of 12 control wells plus 3 standard deviations.

Results: When aptamers were coated on the solid phase and antibodies (8G8 or F89) were used as detection reagents, aptamer 1-2, 3-10OH, and 3-59 worked best. These aptamers were able to detect down to 16 ng/ml of recombinant PrPC. The said aptamers also detected PrPC in plasma at 1:10 dilution. When aptamers were used as detection reagents, aptamer 3-10 detected 16 ng/ml of PrPP C while aptamer 1-2 detected 64 ng/ml. Aptamers selected against PrPSc bound to PrPC 23-231 only marginally. Their binding sites may have competed with monoclonal antibodies in this assay format. Other ligand combinations with fluorescence are being investigated to improve sensitivity of the detection.

### Example 5

### Evaluation of Aptamer Binding Kinetics Using Surface Plasmon Resonance Imaging

Binding kinetics of biotinylated PrPP C-specific aptamer 3-10 to recombinant 23-1231 molecule was evaluated using surface plasmon resonance imaging (Reichert Inc., NY). This experiment was performed in Pall Corporation, Ann Arbor, Mich. A gold surface was activated with EDS-NHS chemistry (as suggested by the manufacturer) to obtain covalent binding of streptavidin. Biotinylated 3-10 was applied to bind to the streptavidin molecule immobilized on the gold surface. Recombinant 23-231 was first applied to study binding kinetics over time. Subsequently, the bound recombinant protein was stripped and plasma was applied at 1:10 dilution to evaluate binding of mammalian PrP (data not shown).

Results: The aptamer 3-10 bound to both recombinant and human PrP at high affinities. The rate of reaction for each isoform of PrP with several aptamers is currently being calculated to identify affinity constants.

The embodiments described above are examples of preferred embodiments and are not intended to limit the scope of the claims set forth below. Variations to the inventions described herein, including alternate embodiments not specifically described, are quiet possible and are encompassed by the claims as understood by one of ordinary skill in the art. Indeed, the claimed inventions have their broad and ordinary meaning as set forth below in the claims.

1. An isolated polynucleotide selected from the group consisting of the nucleic acids shown in Tables 1 and 3, and in FIGS. 8 and 9.

2. An isolated polynucleotide according to claim 1, wherein the isolated polynucleotide is a ligand for PrP or for a fragment or derivative thereof.

3. An isolated polynucleotide according to claim 2, wherein the isolated polynucleotide is a ligand for PrP^{C} or for a fragment or derivative thereof.

4. An isolated polynucleotide according to claim 1, wherein the isolated polynucleotide comprises a 5′ overhang.

5. An isolated polynucleotide according to claim 1, wherein the isolated polynucleotide comprises a 3′ overhang.

6. An isolated polynucleotide according to claim 1, wherein the isolated polynucleotide comprises a 5′ and 3′ overhang.

7. An isolated polynucleotide according to claim 4, wherein the 5′ overhang comprises at least 4 nucleotides.

8. An isolated polynucleotide according to claim 7, wherein the 5′ overhang comprises 5′-CTTA-3′.

9. An isolated polynucleotide according to claim 5, wherein the 3′ overhang comprises at least 4 nucleotides.

10. An isolated polynucleotide according to claim 9, wherein the 3′ overhang comprises 5′-AATT-3′.

11. An isolated polynucleotide according to claim 6, wherein the 5′ and 3′ overhangs each comprise at least 4 nucleotides.

12. An isolated polynucleotide according to claim 11, wherein the 5′ overhang comprises 5′-CTTA-3′ and the 3′ overhang comprises 5′-AATT-3′.

13. An isolated polynucleotide according to claim 4, wherein the 5′ overhang comprises 5′-TTT GGT CCT TGT CTT ATG TCC AGA ATG C-3′ (SEQ ID NO: 3).

14. An isolated polynucleotide according to claim 5, wherein the 3′ overhang comprises 5′-ATT TCT CCT ACT GGG ATA GGT GGA TTA T-3′ (SEQ ID NO: 4).

15. An isolated polynucleotide according to claim 6, wherein the 5′ overhang comprises 5′-TTT GGT CCT TGT CTT ATG TCC AGA ATG C-3′ (SEQ ID NO: 3) and the 3′ overhang comprises 5′-ATT TCT CCT ACT GGG ATA GGT GGA TTA T-3′ (SEQ ID NO: 4).

16. A method for detecting a PrP comprising contacting a sample with an isolated polynucleotide selected from the group consisting of the nucleic acids shown in Tables 1 and 3, and in FIGS. 8 and 9 and determining if there is binding of said isolated polynucleotide and PrP.

17. The method according to claim 16, wherein said sample is contacted with an isolated polynucleotide selected from the group consisting of the nucleic acids shown in Tables 1 and 3, and in FIGS. 8 and 9 and determining if there is binding of said isolated polynucleotide and PrP^{C}.
